(19) United States
(12) Patent Application Publication
(10) Pub. No.: US 2017/0072074 A1
(43) Pub. Date: Mar. 16, 2017

(54) IMPLANTABLE CELL ENCAPSULATION DEVICE

(71) Applicant: GLUSENSE LTD., Rehovot (IL)

(72) Inventors: Micha GLADNIKOFF, Tel Aviv (IL); Boaz BRILL, Rehovot (IL); Itamar WEISMAN, Karkour (IL)

(73) Assignee: GLUSENSE LTD., Rehovot (IL)

(21) Appl. No.: 15/360,523

(22) Filed: Nov. 23, 2016

Related U.S. Application Data
(63) Continuation-in-part of application No. 14/758,493, filed on Jun. 29, 2015.
(60) Provisional application No. 62/258,783, filed on Nov. 23, 2015.
(60) Provisional application No. 61/746,691, filed on Dec. 28, 2012.

(51) Int. Cl. A61K 49/00; A61B 5/145; A61B 5/1459; A61B 5/00

(52) U.S. Cl. CPC A61K 49/0097; A61B 5/0071; A61B 5/14532; A61B 5/1459; A61K 49/0045; A61B 2562/162; A61B 2562/0233

(57) ABSTRACT

Apparatus is provided for implantation of live cells in a subject. The apparatus includes an implantable immunoisolation device, which includes (a) a chamber, which contains the live cells; (b) an inner membrane layer, which is disposed at an external surface of the chamber, and which comprises a selective membrane that is permeable to nutrients; and (c) an outer hydrogel layer, which comprises a hydrogel, and which is attached to and coats an outer surface of the inner membrane layer. Other embodiments are also described.

### CROSS-REFERENCE TO RELATED APPLICATIONS

The present patent application (a) claims the benefit of U.S. Provisional Application 62/258,783, filed Nov. 23, 2015, and (b) is a continuation-in-part of U.S. application Ser. No. 14/758,493, filed Jun. 29, 2015, in the US national stage of International Application PCT/IB2013/061368, filed Dec. 27, 2013, which claims priority from U.S. Provisional Application 61/746,691, filed Dec. 28, 2012. All of the above-mentioned applications are assigned to the assignee of the present application and are incorporated herein by reference.

### FIELD OF THE APPLICATION

The present invention relates generally to implantable medical devises, and specifically to implantable medical devices that contain cells.

### BACKGROUND OF THE APPLICATION

The monitoring of various medical conditions often requires measuring the levels of various components within the blood. In order to avoid invasive repeated blood drawing, implantable sensors aimed at detecting various components of blood in the body have been developed. More specifically, in the field of endocrinology, in order to avoid repeated “finger-sticks” for drawing blood to assess the levels of glucose in the blood in patients with diabetes mellitus, implantable glucose sensors have been discussed.

One method for sensing the concentration of an analyte such as glucose relies on Fluorescence Resonance Energy Transfer (FRET). FRET involves the transfer of non-photonic energy from an excited fluorophore (the donor) to another fluorophore (the acceptor) when the donor and acceptor molecules are in close proximity to each other. FRET enables the determination of the relative proximity of the molecules for investigating, for example, the concentration of an analyte such as glucose.

U.S. Pat. No. 7,951,357 to Gross et al., which is incorporated herein by reference, describes a protein which includes a glucose binding site, cyan fluorescent protein (CFP), and yellow fluorescent protein (YFP). The protein is configured such that binding of glucose to the glucose binding site causes a reduction in a distance between the CFP and the YFP. Apparatus is described for detecting a concentration of a substance in a subject, the apparatus comprising a housing adapted to be implanted in the subject. The housing comprises a fluorescence resonance energy transfer (FRET) measurement device and cells genetically engineered to produce, in situ, a FRET protein having a FRET complex comprising a fluorescent protein donor, a fluorescent protein acceptor, and a binding site for the substance.

PCT Publication WO 2014/102743 to Brill et al., which is incorporated herein by reference, describes apparatus for detecting a concentration of an analyte in a subject, the apparatus configured to be implanted in a body of the subject and comprising an optical waveguide having a proximal end and a distal end, and a sensing unit disposed at the distal end of the optical waveguide and configured to detect the analyte. The sensing unit comprises at least a first chamber; at least a second chamber disposed around the first chamber at least at a proximal end portion of the first chamber; and live cells that are genetically engineered to produce, in a body of the subject, a sensor protein having a binding site for the analyte, the live cells being disposed within at least one chamber selected from the group consisting of: the first chamber and the second chamber. Other applications are also described.

### SUMMARY OF THE APPLICATION

In some embodiments of the present invention, devices are provided for the encapsulation of live cells in the body of a subject. The devices are configured to maintain high viability of the cells, by enabling an ample supply of nutrients to pass from the body, and minimizing the body's immune response to the cells.

In some applications of the present invention, an implantable immunoisolation device is provided for implantation of live cells in a subject. The device comprises:
a chamber, which contains the live cells;
an inner (lower) membrane layer, which is disposed at an external surface of the chamber, and which comprises a selective membrane that is permeable to nutrients; and
an outer (upper) hydrogel layer, which comprises a hydrogel, and which is attached to and coats the inner membrane layer.

The outer hydrogel layer may serve to minimize foreign body response in the interface between the implantable immunoisolation device and the body, specifically in the area of the inner membrane layer, which is most important because it is the interface that provides nutrients to the cells and allows free passage of metabolites, such as glucose, for measurement.

For some applications, the implantable immunoisolation device comprises a frame, which is shaped so as to define the chamber, and the outer hydrogel layer is flush with an outer surface of the frame at least partially along an interface between the outer hydrogel layer and the frame. This disposition of the outer hydrogel layer generally minimizes the likelihood of the outer hydrogel layer peeling off of the frame, such as during insertion of the immunoisolation device into the subject's body.

For some applications, the implantable immunoisolation device further comprises a non-biodegradable scaffold, and a portion of the hydrogel is disposed in the scaffold, such that the scaffold helps hold the outer hydrogel layer attached to the outer surface of the inner membrane layer. For some applications, the scaffold is shaped so as to define a plurality of lateral walls, which define a plurality of compartments that are open at outer and inner sides (i.e., at the top and bottom), and the portion of the hydrogel is disposed in the compartments.

For some applications, the implantable immunoisolation device is used to encapsulate live cells for sensing a level of a metabolite, such as glucose or other molecules, or for therapeutic applications, such as secretion of a hormone, e.g., insulin or a growth hormone.

There is therefore provided, in accordance with an application of the present invention, apparatus for implantation of live cells in a subject, the apparatus including an implantable immunoisolation device, which includes:

a chamber, which contains the live cells;

an inner membrane layer, which is disposed at an external surface of the chamber, and which includes a selective membrane that is permeable to nutrients; and

an outer hydrogel layer, which includes a hydrogel, and which is attached to and coats an outer surface of the inner membrane layer.

For some applications, the implantable immunoisolation device further includes a non-biodegradable scaffold, and a portion of the hydrogel is disposed in the scaffold, such that the scaffold helps hold the outer hydrogel layer attached to the outer surface of the inner membrane layer.

For some applications:

at least a portion of an inner surface of the scaffold is disposed over the inner membrane layer,

at least 75% of the at least a portion of the inner surface of the scaffold is a non-contacting inner surface that does not directly contact the outer surface of the inner membrane layer,

the portion of the hydrogel disposed in the scaffold is a first portion of the hydrogel, and

a second portion of the hydrogel is disposed between a height of the non-contacting inner surface and the outer surface of the inner membrane layer.

For some applications, 100% of the inner surface of the scaffold does not directly contact the outer surface of the inner membrane layer.

For some applications, an average distance between the inner surface of the scaffold and the outer surface of the inner membrane layer is between 20 and 300 microns.

For some applications:

at least a portion of an inner surface of the scaffold is disposed in direct contact with the second portion of the hydrogel, and has a first surface area,

the outer surface of the inner membrane layer coated by the outer hydrogel layer has a second surface area, and

the first surface area equals between 5% and 30% of the second surface area.

For some applications:

the implantable immunoisolation device includes a frame, which is shaped so as to define the chamber, and

the scaffold is attached to the frame.

For some applications, the scaffold is shaped so as to define a plurality of lateral walls.

For some applications, the lateral walls define a plurality of compartments, which are open at outer and inner sides, and the portion of the hydrogel is disposed in the compartments.

For some applications, the lateral walls define between 4 and 20 compartments.

For some applications, each of the compartments has a surface area of between 0.25 mm2 and 4 mm2.

For some applications, the lateral walls have an average height of between 25 and 300 microns.

For some applications, a largest circular disc that can fit between the lateral walls, while the circular disc is oriented parallel to the inner membrane layer, has a diameter of between 0.5 and 3 mm.

For some applications, an outer surface of the outer hydrogel layer is disposed between 50 microns inwardly from and 50 microns outwardly from an outer surface of the scaffold. For some applications, the outer surface of the outer hydrogel layer is disposed flush with the outer surface of the scaffold.

For some applications, the scaffold is more rigid than the hydrogel.

For some applications, the scaffold includes a porous structure. For some applications, the porous structure includes an element selected from the group consisting of: a mesh, a net, and a fabric.

For some applications, the scaffold is fixed to the inner membrane layer.

For some applications:

the implantable immunoisolation device includes a frame, which is shaped so as to define the chamber, and

the outer hydrogel layer is flush with an outer surface of the frame at least partially along an interface between the outer hydrogel layer and the frame.

For some applications, the outer hydrogel layer is flush with the outer surface of the frame along at least 10% of a length of the interface between the outer hydrogel layer and the frame. For some applications, the outer hydrogel layer is flush with the outer surface of the frame entirely along the interface between the outer hydrogel layer and the frame.

For some applications, the outer hydrogel layer is flat. For some applications, the outer hydrogel layer is cylindrical.

For some applications, the implantable immunoisolation device includes a frame, which is shaped so as to define the chamber, and the inner membrane layer is directly connected to the frame with no intermediate material.

For some applications, the apparatus includes a frame, which is shaped so as to define the chamber, and the inner membrane layer is directly connected to the frame without being glued to the frame.

For some applications, the outer hydrogel layer is chemically attached to the inner membrane layer. For some applications, the outer hydrogel layer is physically attached to the inner membrane layer.

For some applications, the hydrogel is non-biodegradable.

For some applications, the outer hydrogel layer covers at least 50% of the external surface of the inner membrane layer. For some applications, the outer hydrogel layer entirely covers the external surface of the inner membrane layer.

For some applications, the membrane includes one or more materials selected from the group of materials consisting of: polysulfone (PS), modified polysulfone (mPS), polyethersulfone (PES), modified polyethersulfone (mPES), polytetrafluoroethylene (PTFE), PVDF, CA, PE, PP, PAN, PEI, PMMA, Cellulose, PEEK, and polyurethane (PU).

For some applications, the hydrogel includes one or more materials selected from the group or materials consisting of: polyethylene glycol (PEG), polyethylene glycol diacrylate (PEG-DA), polyethylene glycol dimethacrylate (PEG-DMA), polyvinyl acrylate (PVA), Polyhydroxyethylmethacrylate (PHEMA), poly-sulfobetaine (SB), poly-carboxybetaine (CB), Poly(2-methacryloyloxyethyl phosphorylcholine (MPC), alginate, chitin, and copolymers thereof.

For some applications, the hydrogel includes the PEG or the PEG derivative having a molecular weight of between 1 and 10 kDa and a concentration of between 3% and 20%.

For some applications, the molecular weight between 2 and 5 kDa, and the concentration is between 6% and 15%.

For some applications, the membrane allows passage of molecules smaller than a first molecular size that is no more than 30 KDa, and blocks passage of molecules larger than a second molecular size that is at least 80 KDa.

For some applications, the first molecular size is no more than 5 KDa, and the second molecular size is at least 150 KDa.

For some applications, a molecular weight cutoff (MWCO) of the membrane is between 10 and 150 kDa.

For some applications, the MWCO of the membrane is between 30 and 70 kDa.

For some applications, the implantable immunoisolation device includes a frame, which is shaped so as to define the chamber, and side walls of the frame that extend along a longest dimension of the frame are inclined.

For some applications:

the implantable immunoisolation device includes a frame, which is shaped so as to define the chamber, and

the outer hydrogel layer is recessed with respect to an outer surface of the frame at least partially along an interface between the outer hydrogel layer and the frame.

For some applications, the outer hydrogel layer is recessed with respect to the outer surface of the frame along at least 10% of a length of the interface between the outer hydrogel layer and the frame.

For some applications, the outer hydrogel layer is recessed with respect to the outer surface of the frame entirely along the interface between the outer hydrogel layer and the frame.

For some applications, the outer hydrogel layer is flat. For some applications, the outer hydrogel layer is cylindrical.

For some applications:

the implantable immunoisolation device includes a frame, which is shaped so as to define the chamber, and the outer hydrogel layer has an angle of contact of less than 45 degrees with an outer surface of the frame at least partially along an interface between the outer hydrogel layer and the frame.

For some applications, the outer hydrogel layer has the angle of contact with the outer surface of the frame along at least 10% of a length of the interface between the outer hydrogel layer and the frame.

For some applications, the outer hydrogel layer has the angle of contact with the outer surface of the frame entirely along the interface between the outer hydrogel layer and the frame.

For some applications, the angle of contact is less than 30 degrees, such as less than 15 degrees.

There is further provided, in accordance with an application of the present invention, a method of manufacturing an implantable device, including:

placing a periphery of a membrane layer against a surface of a frame that is shaped so as to define a chamber; and

attaching the membrane layer to the frame by, using a hot surface of a manufacturing tool, pressing the periphery of the membrane layer against the surface of the frame.

For some applications, placing the periphery includes placing the periphery against a recessed plane defined by the frame.

For some applications, placing the periphery includes placing the periphery against an external surface of the frame.

For some applications, the hot surface of the manufacturing tool is shaped as a ridge that protrudes from the manufacturing tool, and pressing includes pressing the ridge against the surface of the frame.

For some applications, the method further includes placing live cells into the chamber.

For some applications, a temperature of the hot surface of the manufacturing tool is between 150 C and 300 C, and pressing includes pressing, using the hot surface, the periphery of the membrane layer against the surface of the frame for between 0.1 and 2 seconds.

There is still further provided, in accordance with an application of the present invention, a method of manufacturing an implantable device, including:

injecting live cells into a chamber defined by a frame of the implantable device, through an opening defined by the frame; and

thereafter, sealing the chamber by placing a plug in the opening and welding the plug to the frame of the device by applying a hot surface of a manufacturing tool and pressing the hot surface against the frame of the device.

For some applications, the temperature of the hot surface of the manufacturing tool is between 150 C and 300 C, and pressing includes pressing the hot surface of the manufacturing tool against the frame of the device for between 0.1 and 2 seconds.

The present invention will be more fully understood from the following detailed description of embodiments thereof, taken together with the drawings, in which:

### BRIEF DESCRIPTION OF THE DRAWINGS

FIGS. 1A-C are schematic illustrations of an implantable immunoisolation device for encapsulation of live cells in a body of a subject, in accordance with an application of the present invention;

FIG. 2A is a schematic cross-sectional view of the implantable immunoisolation device of FIGS. 1A-C, accordance with an application of the present invention;

FIG. 2B is a schematic cross-sectional illustration of another configuration of the implantable immunoisolation device of FIGS. 1A-C, in accordance with an application of the present invention;

FIG. 3 is a schematic cross-sectional illustration of another configuration of the implantable immunoisolation device of FIGS. 1A-C, in accordance with an application of the present invention;

FIG. 4 is a schematic cross-sectional illustration of yet another configuration or implantable immunoisolation device of FIGS. 1A-C, in accordance with an application of the present invention;

FIG. 5 is a schematic cross-sectional illustration of still another configuration of implantable immunoisolation device of FIGS. 1A-C, in accordance with an application of the present invention;

FIG. 6 is a schematic cross-sectional illustration of another configuration of the implantable immunoisolation device of FIGS. 1A-C implanted subcutaneously in a subject, in accordance with an application of the present invention;

FIG. 7 is a schematic cross-sectional illustration of another implantable immunoisolation device, in accordance with some applications of the present invention;

FIG. 8 is a schematic cross-sectional illustration of apparatus for facilitating cell growth, in accordance with some applications of the present invention;

FIG. 9 is a schematic cross-sectional illustration of a multi-layer immunoisolation system, in accordance with an application of the present invention;

FIG. 10 is a schematic cross-sectional illustration of another multi-layer immunoisolation system, in accordance with an application of the present invention; and

FIGS. 11A-C are schematic illustrations of another implantable immunoisolation device for encapsulation of live cells in a body of a subject, in accordance with an application of the present invention;

FIGS. 12A-D are cross-sectional views of the device of FIGS. 11A-C taken along lines XIIA-XIIA, XIIB-XIIB, XIIC-XIIC, and XIID-XIID of FIG. 11B, respectively, in accordance with an application of the present invention; and

FIG. 13 is a schematic cross-sectional illustration of another implantable immunoisolation device for encapsulation of live cells in a body of a subject, in accordance with an application of the present invention.

### DETAILED DESCRIPTION OF APPLICATIONS

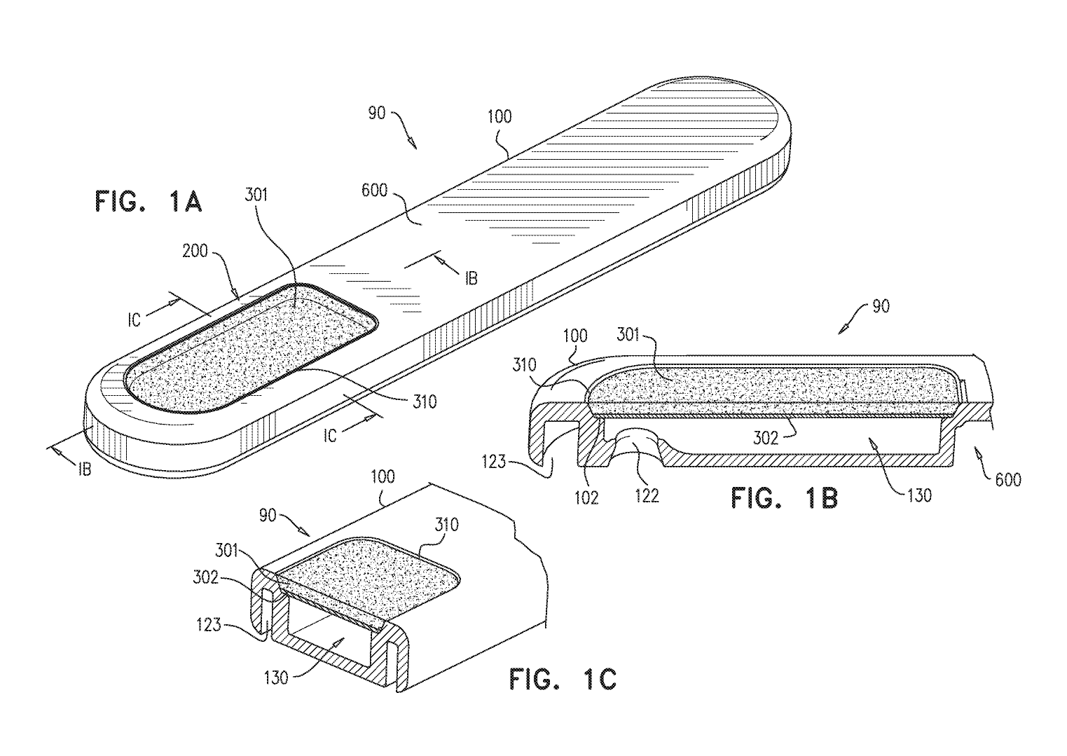

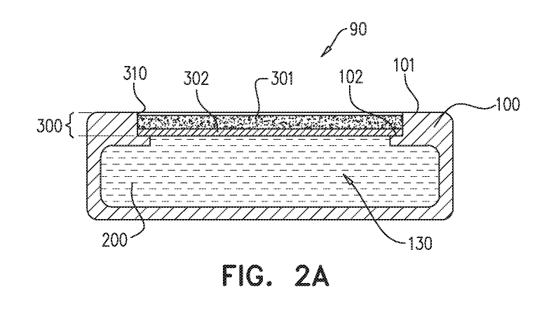

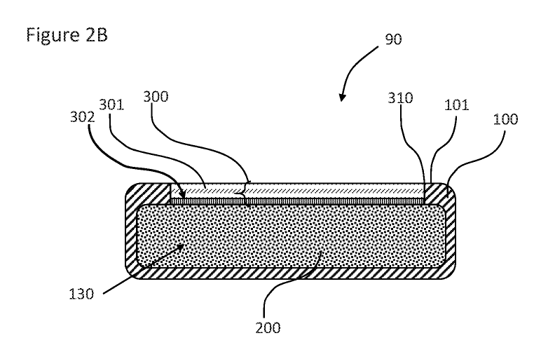

FIGS. 1A-C are schematic illustrations of an implantable immunoisolation device 90 for encapsulation of live cells in a body of a subject, in accordance with an application of the present invention. FIGS. 1B and 1C are cross-sectional views of device 90 taken along lines IB-IB and IC-IC of FIG. 1A, respectively. FIG. 2A is another cross-sectional view of device 90, also taken along line IC-IC of FIG. 1A. FIG. 2B is a schematic cross-sectional view of another configuration of device 90, as described hereinbelow with reference to FIG. 2B.

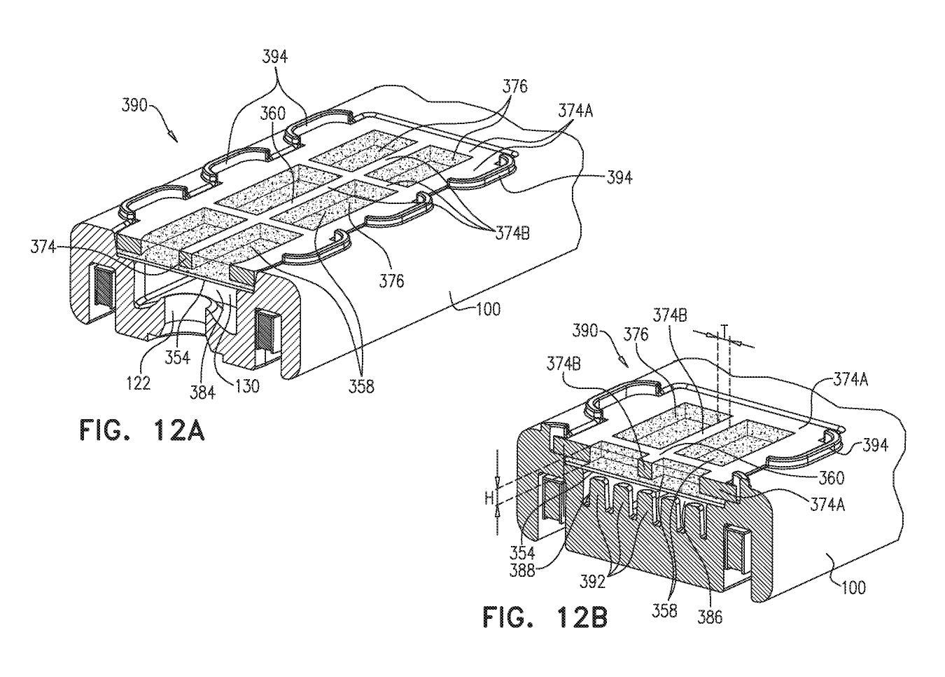

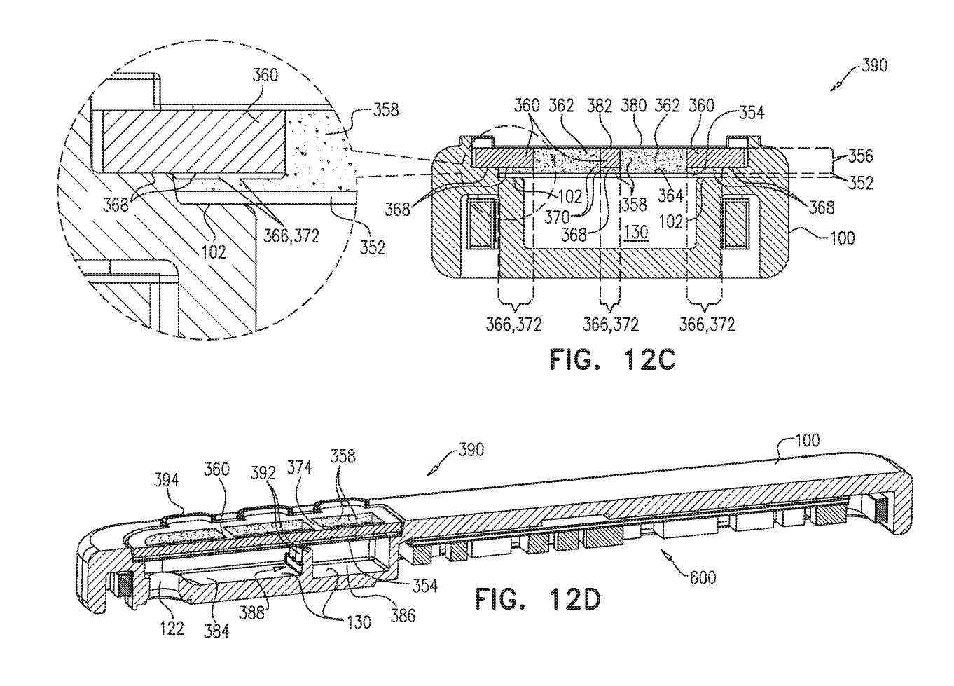

Device 90 comprises a frame 100, which is shaped so as to define a chamber 130 (which optionally is shaped so as to define two or more sub-chambers, such as shown in FIG. 12D, and described hereinbelow in detail with reference to FIGS. 11A and 12D). Chamber 130 contains live cells 200. Typically, frame 100 is optically transparent. Typically, frame 100 comprises a biocompatible plastic (e.g., thermoplastic) material, such as polyethylene, polysulfone, polyethersulfone (PES), modified polyethersulfone (mPES), polyurethane (PU), poly(methyl methacrylate) (PMMA), polytetrafluoroethylene (PTFE), polycarbonate, polyethylene terephthalate (PET), polyaryletheretherketone (PEEK), or polyethylenimine (PEI). For some applications, the bottom of device 90 is sealed with an epoxy.

For some applications, device 90 comprises an electronics compartment 600, which typically comprises one or more light sources and/or circuitry, such as described in the above-mentioned U.S. Pat. No. 7,951,357. For some applications, device 90 has dimensions of approximately 3 mm×1 mm×20 mm.

For some applications, cells 200 are genetically engineered to produce, in situ, a fluorescent biosensor protein, such as a FRET protein, for example using techniques described in the above-mentioned U.S. Pat. No. 7,951,357; for some of these applications, chamber 130 further contains the fluorescent biosensor protein.

Device 90 further comprises a multi-layer tissue interface 300, which is disposed so as to separate between chamber 130 and the body of the subject. Multi-layer tissue interface 300 typically comprises:
an inner (lower) membrane layer 302, which is disposed at an external surface of chamber 130, and which comprises a selective membrane that is permeable to nutrients; and
an outer (upper) hydrogel layer 301, which comprises a hydrogel, and which is attached to and coats an outer (upper) surface of inner membrane layer 302.

In the configuration shown in FIGS. 1A-C, 2A, 5, and 6, frame 100, around inner membrane layer 302, is shaped so as to define a recessed plane 102, above and against which inner membrane layer 302 is disposed. In the configurations shown in FIGS. 2B, 3, and 4, frame 100 does not define recessed plane 102.

For some applications, outer hydrogel layer 301 is shaped so as to minimize the likelihood of outer hydrogel layer 301 peeling off of frame 100, such as during insertion of immunoisolation device 90 into the subject's body.

To this end, for some applications, such as shown in FIGS. 2B, 3, 5, and 6, outer hydrogel layer 301 is flush with an outer (upper) surface 101 of frame 100 at least partially along an interface 310 between outer hydrogel layer 301 and frame 100, e.g., at least along a portion of interface 310 disposed in a leading direction of device 90 during insertion of the device into the subject's body. For some applications, outer hydrogel layer 301 is flush with outer surface 101 of frame 100 along at least 10% of a length of interface 310 between outer hydrogel layer 301 and frame 100, such as along at least 25%, e.g., at least 50%, of the length. For example, outer hydrogel layer 301 may be flush with outer surface 101 of frame 100 entirely along interface 310 between outer hydrogel layer 301 and frame 100, such as shown in the figures. As used in the present application, including in the claims, “flush” means even or level, as with a surface; forming the same surface, such as the same plane or cylindrical surface.

For some applications, such as shown in FIGS. 1A-C, 2A-B, 5, and 6, outer hydrogel layer 301 is flat.

For some applications, during manufacture of device 90, the hydrogel is cast in such a manner that the hydrogel does not swell over the height of frame 100, while still filling the entire space defined by inner membrane layer 302 and frame 100.

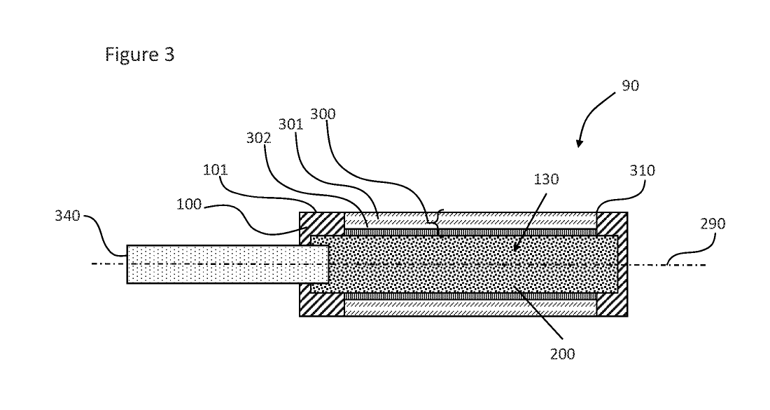

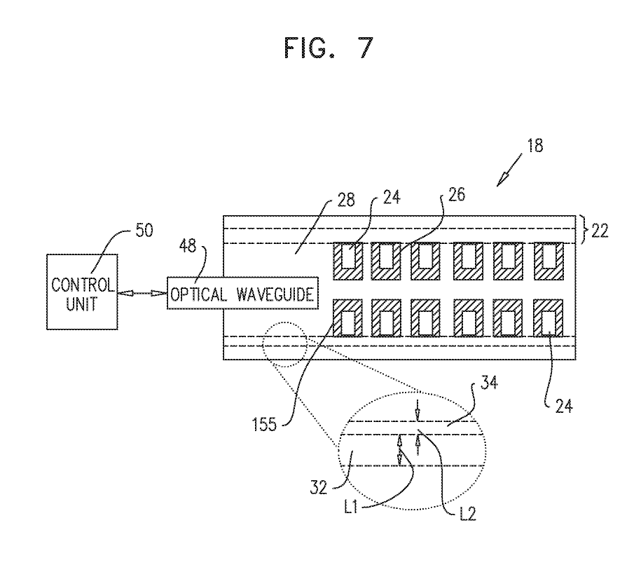

Reference is now made to FIG. 3, which is a schematic cross-sectional illustration of another configuration of implantable immunoisolation device 90, in accordance with an application of the present Invention. In this configuration, device 90 is cylindrical about a longitudinal axis 290 of device 90, and typically comprises an optical waveguide 340, such as described hereinbelow with reference to FIG. 7, mutatis mutandis. For some applications, such as shown in FIG. 3, outer hydrogel layer 301 is flush with outer surface 101 of frame 100 at least partially along interface 310 between outer hydrogel layer 301 and frame 100. Outer hydrogel layer 301 is thus cylindrical (as a result, outer hydrogel layer 301 is typically flush with the outer diameter of device 90).

For some applications, such as shown in FIGS. 1A-C and 2A, outer hydrogel layer 301 is recessed with respect to outer surface 101 of frame 100 at least partially along interface 310 between outer hydrogel layer 301 and frame 100, such as along at least 10% of a length of interface 310, e.g., at least 25%, such as at least 50%. For some applications, outer hydrogel layer 301 is recessed with respect to outer surface 101 of frame 100 entirely along interface 310 between outer hydrogel layer 301 and frame 100. For some applications, outer hydrogel layer 301 is flat. For other applications, outer hydrogel layer 301 is cylindrical.

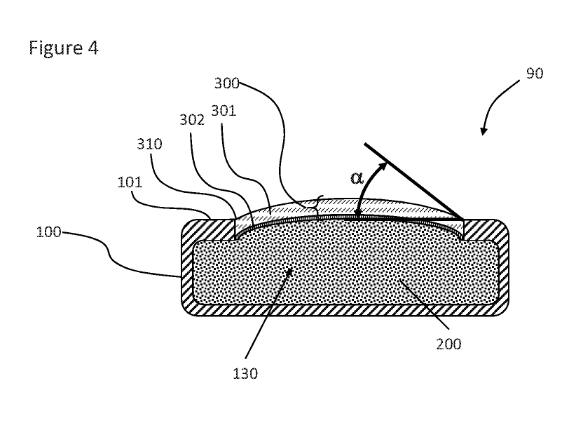

Reference now made to FIG. 4, which is a schematic cross-sectional illustration of yet another configuration of implantable immunoisolation device 90, in accordance with an application of the present invention. In this configuration, outer hydrogel layer 301 has an angle of contact α (alpha) of less than 45 degrees, such as less than 30 degrees, e.g., less than 15 degrees, with outer surface 101 of frame 100 at least partially along interface 310 between outer hydrogel layer 301 and frame 100, such as along at least 10% of a length of interface 310, e.g., at least 25%, such as at least 50%. For some applications, outer hydrogel layer 301 has the angle of contact with outer surface 101 of frame 100 entirely along interface 310 between outer hydrogel layer 301 and frame 100. Thus, in this configuration, outer hydrogel layer 301 (and, typically, inner membrane layer 302) protrude from outer surface 101 of frame 100.

Typically, outer hydrogel layer 301 covers at least 50% of the external surface of inner membrane layer 302, such as at least 80%, e.g., at least 90%, such entirely covers the external surface of inner membrane layer 302. Typically, outer hydrogel layer 301 has an average thickness of less than 500 microns, e.g., less than 300 microns, such as less than 200 microns, e.g., less than 100 microns, and/or of at least 50 microns.

The membrane of inner membrane layer 302 prevents entry into chamber 130 of large proteins and/or cells, while allowing entry of small molecule including nutrients to support cell viability. (“Nutrients,” in the context of the specification and in the claims, includes oxygen, glucose, and other molecules important for cell survival.) Typically, a molecular weight cutoff (MWCO) of the membrane is less than 150 kDa, such as less than 100 kDa, less than 70 kDa, less than 50 kDa, less than 30 kDa, or less than 10 kDa, and/or at least 10 kDa, such as at least 30 kDa.

For some applications, the membrane comprises a hydrophilic polymer or hydrophobic polymer with hydrophilic surface modification. For some applications, the membrane comprises one or more of the following materials: polysulfone (PS), modified polysulfone (mPS), polyethersulfone (PES), modified polyethersulfone (mPES), polytetrafluoroethylene (PTFE), Polyvinylidene fluoride (PVDF), CA, polyethylene (PE), polypropylene (PP), Polyacrylonitrile (PAN), polyethylenimine (PEI), poly(methyl methacrylate) (PMMA), Cellulose, Polyether ether ketone (PEEK), and polyurethane (PU).

For some applications, the hydrogel of outer hydrogel layer 301 is non-biodegradable. For example, the hydrogel may be synthetic (i.e., not containing biological molecules (endotoxins)), e.g., of the PEG-family, including linear and branched macromolecules, e.g. PEG-DA, PEG-DMA; from the pHEMA-family; PVA; a zwitterionic hydrogel e.g. poly-sulfobetaine (SB), poly-carboxybetaine (CB) or Poly(2-methacryloyloxyethyl phosphorylcholine) (MPC); copolymers thereof. For some applications, the hydrogel comprises the PEG or the PEG derivative having a molecular weight of between 1 and 10 kDa (e.g., between 2 and 5 kDa), and a concentration of between 3% and 20% (e.g., between 6% and 15%). Alternatively, the hydrogel may be derived from biological source, e.g., comprises a polysugar such as alginate, chitin, or copolymers thereof.

For other applications, the hydrogel has controlled degradability. For example, the hydrogel may be synthetic (i.e., not containing organic molecules), and controlled degradability is achieved by inserting sites for hydrolysis. Alternatively, the hydrogel may be derived from a biological source, e.g., polysugars, e.g., glucosaminoglycans, e.g., hyaluronic acid; proteins, e.g., collagen or gelatin; extracellular matrix; and copolymers thereof. Any combination (co-polymer) of two or more of the above-mentioned hydrogels may also be used.

For some applications, the membrane allows passage of molecules smaller than a first molecular size and blocks passage of molecules larger than a second molecular size. For example, the first molecular size may be no more than 30 KDa (e.g., no more than 15 KDa), and the second molecular size may be at least 50 KDa (e.g., at least 80 KDa), e.g., the first molecular size may be no more than 5 KDa, and the second molecular size may be at least 100 KDa (e.g., at least 150 KDa).

Reference is made to FIGS. 1A-4. For some applications, a thin layer (e.g., having a thickness of less than 10 micron, such as less than 1 micron, e.g., less than 2.5 nm) of a highly biocompatible material, e.g., poly(ethylene glycol) (PEG), is applied over portions of outer surface 101 of frame 100, excluding over outer hydrogel layer 301. For example, the layer may be applied as a molecular “brush” in which one end of each polymer molecule is attached to the surface. For example, PEG molecules having a molecular weight of between 2 and 20 kDa may be used in such a molecular brush. The layer may minimize foreign body response of the tissue, e.g., by minimizing attachment of proteins to the surface, by providing a highly hydrophilic outer surface.

Reference is still made to FIGS. 1A-4. For some applications, inner membrane layer 302 is directly connected to frame 100 with no intermediate material, including any type of glue (i.e., inner membrane layer 302 is directly connected to frame 100 without being glued to frame 100).

Reference is still made to FIGS. 1A-4. For some applications, inner membrane layer 302 is attached to frame 100 by:
placing a periphery of inner membrane layer 302 against a surface of frame 100, such as recessed plane 102, or an external surface of frame 100, and
using a hot a surface of a manufacturing tool, pressing the periphery of inner membrane layer 302 against the surface of frame 100.

For some applications, the hot surface of the manufacturing tool is shaped as a ridge that protrudes from the manufacturing tool, such that only the ridge contacts inner membrane layer 302. As a result, an indentation is typically formed in the periphery of inner membrane layer 302, either at the edge of the inner membrane layer or near the edge, e.g., within 200 microns, such as 100 microns, e.g., 50 microns of the edge. For some applications, a temperature of the hot surface of the manufacturing tool is between 150 C and 300 C, and/or the periphery of inner membrane layer 302 is pressed, using the not surface, against the surface of frame 100 for between 0.1 and 2 seconds. These techniques may be used either in combination with outer hydrogel layer 301, as described herein, or for attaching a membrane to a cell encapsulation device that does riot comprise outer hydrogel layer 301.

Alternatively, inner membrane layer 302 is attached. to frame 100 using other forms of application of heat, e.g., laser welding or ultrasound.

Reference is still made to FIGS. 1A-4. For some applications, outer hydrogel layer is chemically attached. to inner membrane layer 302, such as by covalent binding. Alternatively or additionally, for some applications, outer hydrogel layer 301 is physically attached to inner membrane layer 302. For example, the hydrogel may penetrate into pores of the membrane while in liquid form and slightly swell upon crosslinking, thus pressing against the sides of the pores, holding the hydrogel in place. For some applications, the hydrogel is configured to have controlled swelling, e.g., to swell less than 25%, such as less than 10%, e.g., less than 5%. For some applications, the hydrogel is configured to have controlled stiffness, such as of 0.5-400 kPa, e.g., 1-100 kPa, such as 4-20 kPa.

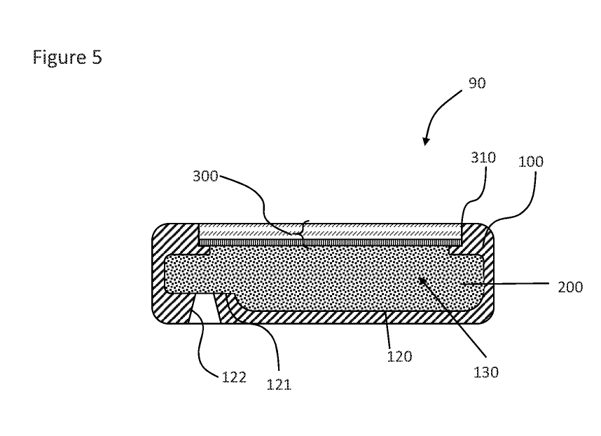

Reference is now made to FIG. 5, which is a schematic cross-sectional illustration of still another configuration of implantable immunoisolation device 90, in accordance with an application of the present invention. The techniques of this configuration may be implemented in combination with the techniques of FIGS. 1A-C, 2A-B, 4, or 6. In this configuration, frame 100 is shaped so as to define an injection opening 122 (e.g., conical) for injection of cells 200. Typically, multi-layer tissue interface 300 is fixed to frame 100 before insertion of cells 200. A needle is subsequently interfaced with injection opening 122; the injection opening provides a surface for tight attachment of the needle. The needle is then used to inject cells 200 through injection opening 122. After injection of the cells, injection opening 122 is self-sealing, or is sealed, such as using a plug (not shown). For some applications, chamber 130 is sealed by placing a plug in injection opening 122 and welding the plug to frame 100 of device 90 by applying a hot surface of a manufacturing tool and pressing the hot surface against the frame of the device. For example, the temperature of the hot surface of the manufacturing tool may be between 150 C and 300 C, and/or the hot surface of the manufacturing tool may be pressed against frame 100 of device 90 for between 0.1 and 2 seconds.

For some applications, as shown, a wall of chamber 130 is thicker surrounding injection opening 122, to provide sufficient thickness to support an opening long enough to seal tightly with the needle, and to resist breakage of the frame because of the pressure that is applied in order to seal between the needle and the injection opening.

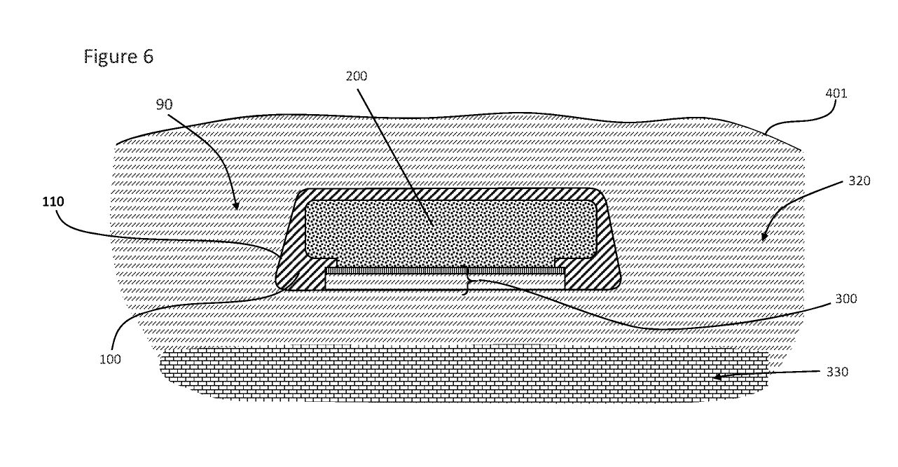

Reference is now made to FIG. 6, which is a schematic cross-sectional illustration of another configuration of implantable immunoisolation device 90 implanted subcutaneously in a subject, in accordance with an application of the present invention. Immunoisolation device 90 is shown implanted in soft skin tissue 320, such as skin or fat, beneath skin 401 and above hard tissue 330, such as muscle, bone, and/or tendon.

For some applications, side walls 110 of frame 100 that extend along a longest dimension of frame 100 are inclined, which may reduce the likelihood of device 90 rotating or flipping during or after implantation. Typically, the inclination is such that the wider side is nearer the harder tissue on the inside of the body; in addition, for some applications, multi-layer tissue interface 300 is disposed on the wider side. For some applications, an angle of the side wall with the wider side is less than 80 degrees, e.g., less than 70 degrees, e.g., less than 60 degrees.

Reference is again made to FIG. 3. In an experiment conducted on behalf of the inventors, the viability of cells in vivo in devices similar to the configuration of device 90 shown in FIG. 3 was demonstrated for over 9 months. The devices were produced using cells that produce a glucose-sensitive fluorescent protein, and PEG-DA for the outer hydrogel layer, and were implanted into a rat animal model. Devices have been retrieved after 6, 13, 19, 26, and 39 weeks from implantation and analyzed for cell metabolism and fluorescent protein expression. Both parameters have shown steady-state performance throughout the period, providing proof-of-principle for the efficiency of the immunomodulation method.

Reference is again made to FIGS. 1B and 1C. In some applications, frame 100 is shaped so as to define a groove 123 for encapsulation of a radiofrequency (RF) receiver coil. Groove 123 typically partially surrounds chamber 130 (such as three sides of chamber 130), but not other portions of frame 100. The RF receiver coil is placed in groove 123 and within electronics compartment 600 near a peripheral wall thereof (and around the circuitry and light source(s) therein). Providing the groove allows for the RF receiver coil to be longer than it would be if it were instead placed entirely within electronics compartment 600. In addition, placement of the RF receiver coil in groove 123, rather than between the electronics compartment and chamber 130, prevents the coil from physically blocking light transmitted from electronics compartment 600 to chamber 130.

One of the challenges in the design of a cell-based implantable device is the maintenance of a significant population of cells over the long term, e.g., over a year or longer. In accordance with some applications of the present invention, techniques are provided for maintaining a desired cell population size over time, including both:
restraining cell population growth, i.e., cell proliferation, in order to avoid over-population that leads to a shortage of nutrients in cells farther from the edge of the device, which would create a necrotic core of cells that eventually intoxicates the entire cell population; and
allowing limited cell proliferation to replace cells that die over time, in order to prevent dwindling of the cell population in the device, which would eventually render the device dysfunctional.

In order to balance the above-mentioned conflicting goals and preserve a generally constant cell population over a long period of time, e.g., at least one year, a three-layer cell encapsulation structure is provided, which comprises a substantially non-degradable three-dimensional scaffold having surfaces to which cells are attached, and a hydrogel, which is applied to the cells.

The scaffold, cells, and hydrogel are arranged such that the cells are sandwiched in spaces between the hydrogel and the surfaces of the scaffold. The cells are arranged in monolayers on at least 50% of an aggregate surface area of the surfaces of the scaffold. This arrangement allows mobility and proliferation of the cells in the spaces between the hydrogel and the surfaces of the scaffold, and prevents the mobility and the proliferation of the cells to locations outside of the spaces between the hydrogel and the surfaces of the scaffold. Cells within the spaces between the hydrogel and the surfaces of the scaffold that die leave a space upon disintegration. The structure provided by the surface of the scaffold on one side and the hydrogel on the other side maintain the patency of this space until one or more neighboring cells proliferate into the space.

Thus, in any local microscopic environment the encapsulation structure comprises a three-layer stack of (a) the surface of the solid scaffold, (b) the cells, and (c) the hydrogel, in this order. The cells at any location are thus generally limited to a monolayer, allowing free mobility and proliferation of the cells within the narrow space between the scaffold and the hydrogel, but preventing any proliferation into the rest of the volume and creation of three-dimensional cell structures.

The scaffold provides a three-dimensional structure with a high aggregate surface area, and high surface-to-volume ratio, which makes efficient use of the three-dimensional volume of the chamber. The surfaces of the scaffold, although often not flat, serve effectively as a two-dimensional substrate for seeding, growth, and attachment of the cells. If the hydrogel were not provided over the monolayer of the cells, the cells typically grow in three dimensions, away from the surfaces to which they are attached. Such three-dimensional growth would generally result in undesirable over-population, as described above. In addition, for many cell types, cell viability and protein expression, including expression of the sensor protein, are significantly enhanced when cells are attached and spread. Thus cells in this configuration will survive longer and function better than suspended cells, e.g., cells suspended in a hydrogel scaffold.

For some applications, the scaffold comprises microcarrier beads, fibers, a rigid structure, or a sponge structure having a plurality of interconnected internal pores.

The encapsulation structure may combine at least three benefits: (a) good cell attachment, leading to better cell viability and expression, lacking in simpler systems that for example use hydrogel as a scaffold, (b) prevention of over-population which often leads to a necrotic core, because of a limited number of cells and open diffusion channels to the cells via the hydrogel, and (c) enablement of cell mobility and proliferation within a two-dimensional culture, thereby enabling long-term steady state population.

In the context of the present application and in the claims, a membrane which is described as “surrounding” an element is to be understood as surrounding the element at least in part. Thus, for example, a membrane that surrounds a chamber may entirely surround the chamber, or may surround the chamber in part (while another portion of the chamber may be covered with something other than the membrane).

FIG. 7 is a schematic cross-sectional illustration of an implantable immunoisolation device 18, in accordance with some applications of the present invention. A scaffold material 28 is shaped to define one or more chambers 155, e.g., implemented as one or more wells 30. Scaffold material 28 is typically but not necessarily cylindrical, e.g., right-circular-cylindrical. Cells 26, such as one or more monolayers of cells 26, are disposed in wells 30 and/or elsewhere on scaffold material 28. The one or more wells can be a plurality of wells, as shown in FIG. 1, or can be a single well (e.g., shaped to define a helix, like a screw-thread). A total surface area of scaffold material 28 upon which the cells are disposed is typically at least 60% of a total surface area of the scaffold which is illuminated when light passes through the optical wavequide.

Typically, scaffold material 28 is optically transparent. Scaffold material 28 may comprise, for example, molded plastic or polystyrene. Excitation light generated by control unit 50 passes through an optical waveguide 48, and enters each well 30 via transparent scaffold material 28. A signal of light of different wavelengths emitted by the sensor proteins is passed by the optical waveguide 48 to control unit 50. Control unit 50 interprets the different wavelengths in the received light signal as indicative of which portion of the sensor proteins have undergone the conformational change, and, therefore, of the concentration of the analyte (e.g., glucose). Typically, scaffold material 28 is rigid.

For some applications, the scaffold is fabricated using 3D printing, and may comprise a biocompatible material, such as MED610.

A membrane structure 22 permeable to nutrients surrounds scaffold material 28 at least in part and is mechanically supported by the scaffold material. Membrane structure 22 may be a simple membrane (e.g., a homogeneous membrane), or a membrane having multiple components, such as a spatially non-homogeneous membrane structure (e.g., as described hereinbelow with reference to FIG. 7).

For some applications, an optical system comprises optical waveguide 48, which is optically coupled to scaffold material 28 (e.g., at least partially disposed within the scaffold material), in order to enable transmission of an optical signal to and from a control unit 50 of the optical system. For some applications, optical waveguide 48 comprises an optical fiber.

Membrane structure 22 in the implementation shown in FIG. 7 comprises (a) a first material 32 of the membrane comprising a biodegradable material (such as a hydrogel), and (b) a second material 34 of the membrane comprising a non-biodegradable material. Materials 32 and 34 may be in any suitable geometrical configuration with respect to each other that provides fluid communication between body fluid of the subject and materials 32 and 34. For example, as shown in FIG. 7, first material 32 is disposed in a first layer that starts out at a thickness L1 of 50-500 microns. The molecular weight cutoff (MWCO) of first material 32 is typically less than 100 kilodaltons, or less than 50 kilodaltons. First material 32 is degraded in the body over a period of time (e.g., within a period of two weeks to six months, in the presence of body fluids), such that the MWCO of membrane structure 22 increases over time. The thickness L2 of a second layer, comprising second material 34, may be greater than, less than, or the same as thickness L1 of first material 32. For example, L2 may be at least 50 microns and/or less than 250 microns. Second material 34 of the membrane structure typically comprises a material such as Polysulfone (PSU), Teflon (pTFE), or polyethersulfone (PES). The second layer is typically but not necessarily disposed between the cells and the first layer. In summary, one or more chambers having isolated cells disposed therein are surrounded at least in part by membrane structure 22.

In some applications of the present invention, a fully-implantable or partially-implantable sensor device comprises apparatus for facilitating cell growth. For some applications, the apparatus comprises a chamber and a membrane that surrounds the chamber at least in part and is permeable to nutrients. Typically, a scaffold comprising a hydrogel or other suitable material is disposed within the chamber, and a plurality of cells is disposed therein. Additionally, at least one nutrient supply compartment is typically disposed within the chamber, and interspersed with the scaffold such that at least 80% of the cells within the cell-growth medium are disposed within 100 microns of the nutrient supply compartment. In this manner, the nutrient supply compartment is positioned within the chamber such that a diffusion path for nutrients is provided, by the nutrient supply compartment, between the membrane and the at least 80% of the cells.

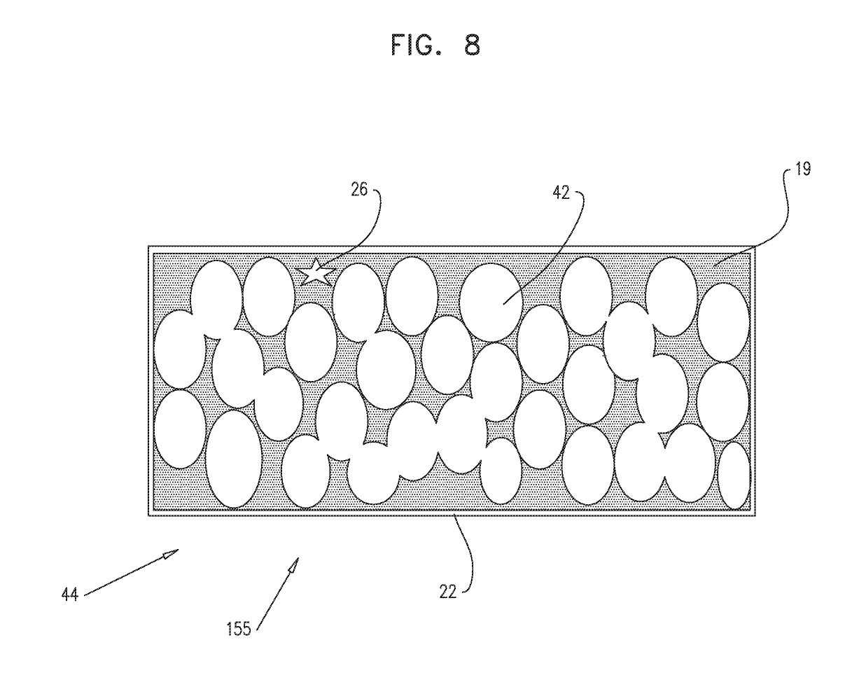

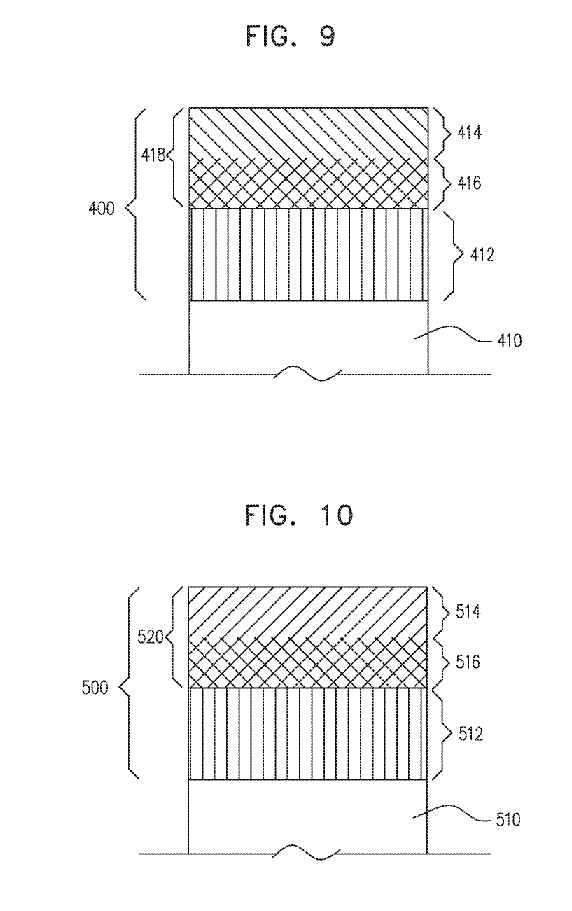

FIG. 8 is a schematic cross-sectional illustration of apparatus 44 for facilitating cell growth, in accordance with some applications of the present invention. Apparatus 44 comprises a chamber 155 for containing the cells, and is typically used in combination with an optical waveguide 48, a control unit 50, and a membrane structure 22, as described with reference to the other figures. For some applications, (a) an optical waveguide is not utilized, or (b) an optical waveguide and a control unit are not utilized. In the depicted application, a scaffold 19 conducive to cell growth (e.g., comprising a hydrogel) typically has at least 1,000 cells 26 (e.g., at least 2,000 cells 26 or at least 5,000 cells 26) and/or less than 30,000 cells 26 (e.g., less than 20,000 cells or less than 10,000 cells 26) disposed therein. Scaffold 19 is typically but not necessarily optically transparent. Typically, the density of the cells is at least 10 million cells/mL and/or less than 30 million cells/mL. A typical volume in which the cells are contained is at least 0.2 microliters and/or less than 2 microliters, e.g., at least 0.5 microliters and/or less than 1 microliter.

At least one nutrient supply compartment comprising a nutrient permeable medium 42 that is arranged to not be conducive to cell growth therein is interspersed with scaffold 19, such that at least 80% of the cells within scaffold 19 are disposed within 100 microns (e.g., within 50 microns) of nutrient permeable medium 42. The nutrient permeable medium is positioned such that an easy diffusion path for nutrients is thus provided, by the nutrient permeable medium, between the subject's body and the at least 80% of the cells.

A volume of the nutrient supply compartment comprising nutrient permeable medium 42 is typically 25%-75% of a volume of chamber 155. Typically, nutrient permeable medium 42 comprises a hydrogel, but in general may comprise any material which suitably diffuses nutrients. The nutrient permeable medium may alternatively or additionally comprises one or more materials such as silicone rubber, fused glass powder, sintered glass powder, a hydrogel, and/or an alginate. This material may be shaped to define one or more spheres, e.g., at least 100 and/or less than 1000 spheres. The volume of chamber 155 is typically at least 20 times (e.g., at least 100 times, e.g., 200-1000 times) a volume of at least one of the spheres. For some applications, the spheres are disposed in the chamber in an efficient packing configuration.

Some applications of the present invention provide a multi-layer immunoisolation system. The viability of cells within a cell-based device strongly depends on an ample supply of oxygen. Generally, the foreign body response following device implantation creates dense fibrotic tissue that encapsulates the device, substantially reducing oxygen diffusion to the device from the blood circulation. Therefore, the viability of cells inside a cell-based device is enhanced by substantial vascularization of the tissue as close as possible to the implanted device, which increases oxygen levels at the device surface. More specifically, for a glucose measurement device, the creation of a dense fibrotic tissue is a potential diffusion barrier for glucose, leading to a time delay between glucose levels in the tissue and glucose levels measured by the device. Such dense fibrotic tissue should thus be avoided in order to maintain the accuracy of the glucose measurement.

The multi-layer immunoisolation system is configured to enhance long-term function of an implanted cell-based device. The multi-layer immunoisolation system comprises at least the following three layers: (a) an inner (lower) membrane layer, which is disposed at an external surface of the device, (b) an outer (upper) neovascularization layer, and (c) a middle protective layer, disposed between the inner membrane layer and the outer neovascularization layer. The multi-layer immunoisolation system comprises a biodegradable scaffold. Before biodegrading, the biodegradable scaffold spans both the outer neovascularization layer and the middle protective layer, such that the outer neovascularization layer comprises a first outer (upper) portion of the biodegradable scaffold, and the middle protective layer comprises a second inner (lower) portion of the biodegradable scaffold. In addition, the middle protective layer further comprises a non-biodegradable hydrogel that impregnates the second inner portion of the biodegradable scaffold. The outer neovascularization layer, which comprises the first outer portion of the biodegradable scaffold, not impregnated with the hydrogel.

The biodegradable scaffold serves at least two functions: (a) during implantation of the device, the biodegradable scaffold protects the soft hydrogel of the middle protective layer from strong shear forces which might otherwise pull off the soft hydrogel; and (b) after implantation of the device, the biodegradable scaffold promotes vascularization of the tissue that grows into the outer neovascularization layer, until the biodegradable scaffold eventually degrades and is totally absorbed.

Upon biodegradation of the biodegradable scaffold, the middle protective layer (now comprising primarily the hydrogel) remains attached to the inner membrane layer. The middle protective layer typically serves to (a) prevent attachment of proteins to the inner membrane layer, thereby minimizing the creation of a fibrotic tissue, and/or (b) repel large proteins, thereby minimizing the fouling of the inner membrane layer. The high water content of the hydrogel of the middle protective layer prevents the attachment of various proteins, so that immune system cells are less likely to attach to the tissue-hydrogel interface, thereby minimizing the overall immune response. As a result of this triple-layer protection, the tissue surrounding the device is characterized by high vascularization and minimal fibrosis.

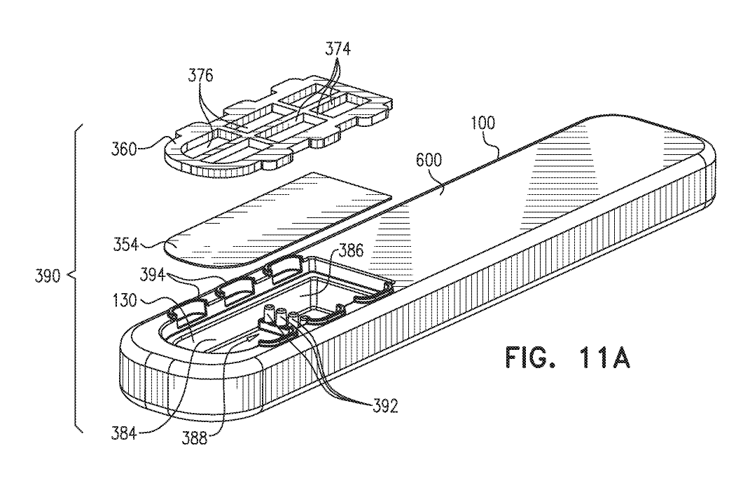

Reference is now made to FIG. 9, which is schematic cross-sectional illustration of a multi-layer immunoisolation system 400, in accordance with an application of the present invention. The viability of cells within a cell-based device strongly depends on an ample supply of oxygen. Generally, the foreign body response following device implantation creates dense fibrotic tissue that encapsulates the device, substantially reducing oxygen diffusion to the device from the blood circulation. Therefore, the viability of cells inside a cell-based device is enhanced by substantial vascularization of the tissue as close as possible to the implanted device, which increases oxygen levels at the device surface. More specifically, for a glucose measurement device, the creation of a dense fibrotic tissue is a potential diffusion barrier for glucose, leading to a time delay between glucose levels in the tissue and glucose levels measured by the device. Such dense fibrotic tissue should thus be avoided in order to maintain the accuracy of the glucose measurement.

Multi-layer immunoisolation system 400 is configured to enhance long-term function of an implantable cell-based device 410. Multi-layer immunoisolation system 400 is disposed at an external surface of device 410. For example, multi-layer immunoisolation system 400 may be integrated into any of the sensing devices described herein instead of, or as an implementation of, external membrane 58.

Multi-layer immunoisolation system 400 comprises at least the following three layers:
a lower (inner) membrane layer 412, which is disposed at an external surface of device 410 (lower membrane layer 412 either is shaped so as to define the external surface of device 410, or is fixed to the external surface of device 410);
an upper (outer) neovascularization layer 414; and
a middle protective layer 416, disposed between lower membrane layer 412 and upper neovascularization layer 414.

Multi-layer immunoisolation system 400 comprises a biodegradable scaffold 416. Before biodegrading, biodegradable scaffold 418 spans both upper neovascularization layer 414 and middle protective layer 416, such that upper neovascularization layer 414 comprises a first upper portion of biodegradable scaffold 418, and middle protective layer 416 comprises a second lower portion of biodegradable scaffold 418.

In addition, middle protective layer 416 further comprises a non--biodegradable hydrogel that impregnates the second lower portion of biodegradable scaffold 418. Upper neovascularization layer 414, which comprises the first upper portion of biodegradable scaffold 418, is not impregnated with the hydrogel.

Biodegradable scaffold 418 serves at least two functions:
during implantation of device 410, biodegradable scaffold 418 protects the soft hydrogel of middle protective layer 416 from strong shear forces which might otherwise pull off the soft hydrogel; and
after implantation or device 410, biodegradable scaffold 418 promotes vascularization of the tissue that grows into upper neovascularization layer 414 (but not into middle protective layer 416), until biodegradable scaffold 418 eventually degrades and is totally absorbed.

Upon biodegradation of biodegradable scaffold 418, middle protective layer 416 (now comprising primarily the hydrogel) remains attached to lower membrane layer 412. Middle protective layer 416 typically serves to (a) prevent attachment of proteins to lower membrane layer 412, thereby minimizing the creation of a fibrotic tissue, and/or (b) repel large proteins, thereby minimizing the fouling of lower membrane layer 412. The high water content of the hydrogel of middle protective layer 416 prevents the attachment of various proteins, so that immune system cells are less likely to attach to the tissue-hydrogel interface, thereby minimizing the overall immune response. Typically, the hydrogel of middle protective layer 416 has a thickness of at least 50 microns, e.g., at least 100 microns, such as in order to enable reactive oxygen species (ROS) decay between inflamed tissue and the device cells. Without the use of the techniques described herein, it 15 generally difficult to attach a hydrogel to a membrane, particularly with a thickness of more than a few microns.

As a result of this triple-layer protection, the tissue surrounding device 410 is characterized by high vascularization and minimal fibrosis.

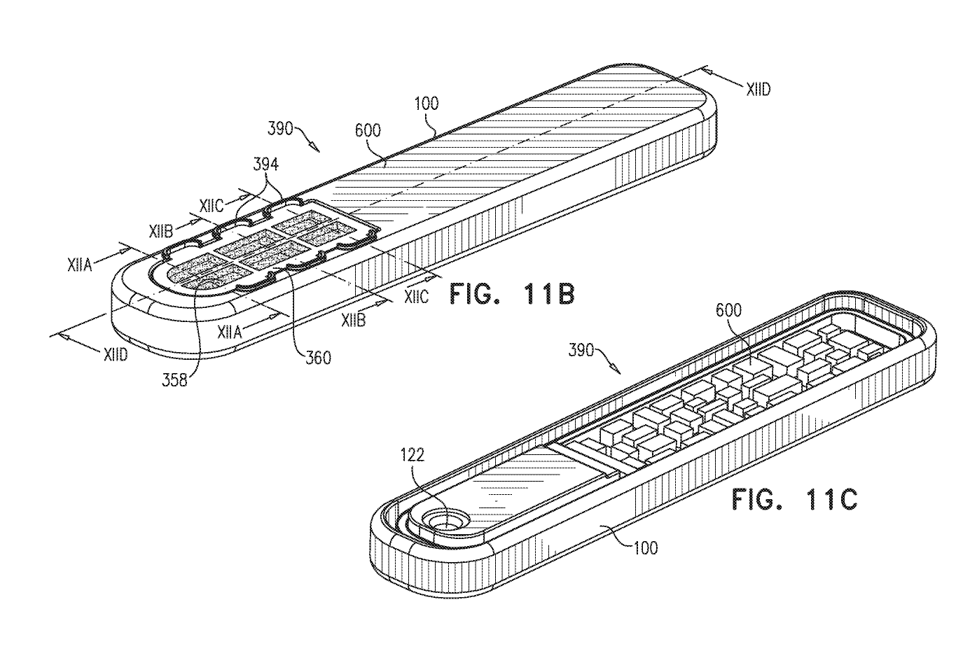

Typically, lower membrane layer 412 (and lower membrane layer 512, described hereinbelow with reference to FIG. 10) has a MWCO of at least 5 KDa, no more than 50 KDa, and/or between 5 and 50 KDa. (Typically, the MWCO of a membrane should be no more than one-third of the size of the molecule to be blocked. Thus, for blocking IgG, which generally has a size of about 150 KDa, a membrane of 50 Ka MWCO or lower should be used.) For some applications, lower membrane layer 412 comprises polysulfone (PS), polyethersulfone (PES), modified polyethersulfone (mPES), or polytetrafluoroethylene (PTFE, Teflon®).

Typically, biodegradable scaffold 418 is highly porous, and has an average pore size of at least 5 microns, no more than 50 microns, and/or or between 5 and 50 microns. For some applications, the scaffold comprises a mesh. Biodegradable scaffold 418 may comprise a polymer, such as polylactic acid (PLA), poly(DL-lactic-co-glycolic acid) (PLGA), poly(3-hydroxypropionic acid) (P(3-HP)), or 3-hydroxypropionic acid (3-HP). Biodegradable polymers and the products of their degradation are typically non-toxic, so as to riot evoke a strong immune response. Additionally, biodegradable polymers typically maintain good mechanical integrity until degraded in order to evoke enhanced vascularization in its vicinity. Finally, biodegradable polymers typically have controlled degradation rates leading to complete disintegration in the body within a few weeks to a few months, which is enough time to evoke vascularization but not become a potential annoyance for the patient a long time after the device is explanted.

Biodegradable scaffold 418 (of upper neovascularization layer 414 and the middle protective layer 416 in combination) typically has a thickness of between 100 and 300 microns and promotes neovascularization by virtue of the large pore size and the slow biodegradation effect. As mentioned above, the scaffold additionally holds the hydrogel layer in place. For some applications, biodegradable scaffold 418 is fixed to the upper (outer) surface of membrane layer 412 by gluing. Alternatively or additionally, for some applications, biodegradable scaffold 418 is fixed to the upper (outer) surface of membrane layer 412 by being directly deposited using electrospinning, i.e., the scaffold is electrospun onto the membrane.

The hydrogel (and hydrogel 520, described hereinbelow with reference to FIG. 10) may comprise poly(ethylene glycol) (PEG), a zwitterionic hydrogel, or any other non-biodegradable hydrogel. The hydrogel is typically impregnated into the second lower portion of biodegradable scaffold 418 in liquid form, and then cross-linked. Applying the hydrogel only to the second lower portion, but not the first upper portion, of biodegradable scaffold 418 may be performed, for example, by (a) impregnating the entire thickness of the biodegradable scaffold, and then drying the hydrogel from the first upper portion, e.g., by soaking the hydrogel into a dry absorbing material, or by a combination of high temperatures and low pressure, or (b) soaking the entire thickness of the biodegradable scaffold with the liquid hydrogel (without a cross-linker), and injecting the cross-linker through lower membrane layer 412, e.g., during application of UV radiation, resulting in preferential crosslinking of the hydrogel from the bottom up; this cross-linking process is halted before the hydrogel in the first upper portion of the biodegradable scaffold is cross-linked, and the remaining hydrogel is washed out of the scaffold.

Some applications of the present invention provide another multi-layer immunoisolation system, which comprises at least the following three layers: (a) a lower (inner) membrane layer, which is disposed at an external surface of the device, (b) an upper (outer) protective layer, and (c) a middle attachment layer, which is disposed between the lower membrane layer and the upper protective layer, and which tightly fixes the upper protective layer to the lower membrane layer. The middle attachment layer comprises a non-biodegradable scaffold, which is tightly fixed to the lower membrane layer, such as by being deposited directly on the membrane using electrospinning.

The multi-layer immunoisolation system comprises a non-biodegradable hydrogel, which spans both the upper protective layer and the middle attachment layer. In other words, the middle attachment layer comprises a first portion of the hydrogel, and the upper protective layer comprises a second portion of the hydrogel. The hydrogel is impregnated in the scaffold of the middle attachment layer, and extends above the scaffold, i.e., in a direction away from the lower membrane, so as to provide the upper protective layer. The upper protective layer does not comprise the scaffold. As a result, the scaffold is not exposed to tissue, thereby reducing the likelihood that the multi-layer immunoisolation system generates an immune response.

The middle attachment layer holds the hydrogel of the upper protective layer in place on the lower membrane layer. The upper protective layer has a smooth upper (outer) surface, which results in low biofouling of the lower membrane layer, allowing the membrane to efficiently diffuse nutrients into the device even after a long implantation period. In addition, the upper protective layer protects the device by presenting a highly biocompatible surface to the tissue.

Reference is now made to FIG. 10, which is schematic cross-sectional illustration of a multi-layer immunoisolation system 500, in accordance with an application of the present invention. Other than as described below, multi-layer immunoisolation system 500 may have any of the characteristics and properties of multi-layer immunoisolation system 400, described hereinabove with reference to FIG. 9.

Multi-layer immunoisolation system 500 is configured to enhance long-term function of an implanted cell-based device 510. Multi-layer immunoisolation system 500 is disposed at an external surface of device 510. For example, multi-layer immunoisolation system 500 may be integrated into any of the sensing devices described herein instead of, or as an implementation of, external membrane 58.

Multi-layer immunoisolation system 500 comprises at least the following three layers:
a lower (inner) membrane layer 512, which is disposed at an external surface of device 510 (lower membrane layer 512 either is shaped so as to define the external surface of device 510, or is fixed to the external surface of device 510);
an upper (outer) protective layer 514; and
a middle attachment layer 516, which is disposed between lower membrane layer 512 and upper protective layer 514, and which tightly fixes upper protective layer 514 to lower membrane layer 512.

Middle attachment layer 516 comprises a non-biodegradable scaffold, which is tightly fixed to lower membrane layer 512, such as by being deposited directly on the membrane using electrospinning, i.e., the scaffold is electrospun onto the membrane. Typically, the scaffold is highly porous, and may comprise, for example, a polymer such as polyurethane, polyvinylidene fluoride (PVDF), or polyethylene terephthalate (PET). Middle attachment layer 516 typically has a thickness of between 50 and 100 microns.

Multi-layer immunoisolation system 500 comprises a non-biodegradable hydrogel 520, which spans both upper protective layer 514 and middle attachment layer 516. In other words, middle attachment layer 516 comprises a first portion of hydrogel 520, and upper protective layer 514 comprises a second portion of hydrogel 520. Hydrogel 520 is impregnated in the scaffold of middle attachment layer 516, and extends above the scaffold, i.e., in a direction away from lower membrane layer 512, so as to provide upper protective layer 514. Upper protective layer 514 does not comprise the scaffold. As a result, the scaffold is not exposed to tissue, thereby reducing the likelihood that multi-layer immunoisolation system 500 generates an immune response.

Middle attachment layer 516 holds the hydrogel of upper protective layer 514 in place on lower membrane layer 512. Upper protective layer 514 has a smooth upper (outer) surface, which results in low biofouling of lower membrane layer 512, allowing the membrane to efficiently diffuse nutrients into device 510 even after a long implantation period. In addition, upper protective layer 514 protects device 510 by presenting a highly biocompatible surface to the tissue. Upper protective layer 514 typically has a thickness of between 50 and 200 microns.

Reference is now made to FIGS. 11A-C and 12A-D, which are schematic illustrations of an implantable immunoisolation device 390 for encapsulation of live cells in a body of a subject, in accordance with an application of the present invention. FIG. 11A shows components of device 390 prior to assembly, FIG. 11B shows the assembled device 390, and FIG. 11C is a bottom-view of device 390 before potting of electronics compartment 600, described hereinbelow, with epoxy. FIGS. 12A-D are cross-sectional views of device 390 taken along lines XIIA-XIIA, XIIB-XIIB, XIIC-XIIC, and XIID-XIID of FIG. 11B, respectively.

Device 390 is similar in some respects to device 90, described hereinabove with reference to FIGS. 1A-6, and may implement any of the features thereof, mutatis mutandis.

Device 390 comprises a multi-layer tissue interface 350, which is disposed so as to separate between chamber 130 and the body of the subject. Multi-layer tissue interface 350, similar in some respects to (a) multi-layer tissue interface 300, described hereinabove with reference to FIGS. 1A-6, (b) multi-layer immunoisolation system 400, described hereinabove with reference to FIG. 9, and (c) multi-layer immunoisolation system 500, described hereinabove with reference to FIG. 10, typically comprises (labeled in FIG. 12C):
an inner (lower) membrane layer 352, which is disposed at an external surface of chamber 130, and which comprises a selective membrane 354 that is permeable to nutrients; and
an outer (upper) hydrogel layer 356, which comprises a hydrogel 358, and which is attached to and coats an outer (upper) surface of inner membrane layer 352.

For some applications, frame 100, around inner membrane layer 352, is shaped so as to define recessed plane 102 (labeled in FIG. 12C), above and against which inner membrane layer 352 is disposed. Alternatively, frame 100 does not define recessed plane 102.

For some applications, outer hydrogel layer 356 is shaped so as to minimize the likelihood of outer hydrogel layer 356 peeling off of frame 100, such as during insertion of immunoisolation device 390 into the subject's body.

To this end, for some applications, immunoisolation device 390 further comprises a non-biodegradable scaffold 360. A first portion 362 of hydrogel 358 (labeled in FIG. 12C) is disposed in scaffold 360, such that scaffold 360 helps hold outer hydrogel layer 356 attached to an outer (upper) surface 364 of inner membrane layer 352, similar in some respects to multi-layer immunoisolation system 500, described hereinabove with reference to FIG. 10. For example, scaffold 360 may effectively divide first portion 362 of hydrogel 358 into smaller units, which are less likely to peel off of frame 100 during Insertion than if hydrogel 358 were a single, larger piece. In other words, providing the scaffold reduces the sizes of hydrogel-only areas exposed to the external surface of the device.

Typically, scaffold 360 is attached to frame 100, such that the frame supports and holds the scaffold in place.

For some applications, at least a portion 366 of an inner surface 368 of scaffold 360 is disposed over inner membrane layer 352. (For example, at some axial locations, such as at cross-section XIIA-XIIA, shown in FIG. 12A, the at least a portion 366 may be the entire inner surface 368 of scaffold 360, while at other axial locations, such as cross-sections XIIB-XIIB and XIIC-XIIC, shown in FIGS. 12B and 12C, the at least a portion 366 may be only a portion of inner surface 368 of scaffold 360, because part of inner surface 368 extends laterally to provide fixation of the scaffold to frame 100.)

For some applications, at least 75% (e.g., 100%, as shown) of the at least a portion 366 of inner surface 368 of scaffold 360 is a non-contacting inner surface that does not directly contact outer surface 364 of inner membrane layer 352. Typically, a second portion 370 of hydrogel 358 (labeled in FIG. 12C) is disposed between a height of the non-contacting inner surface of scaffold 360 and outer surface 364 of inner membrane layer 352. Thus, some of second portion 370 is disposed below (inwardly from) the non-contacting inner surface of scaffold 360, and some of second portion 370 is disposed below (inwardly from) the height of the non-contacting inner surface of scaffold 360, below (inwardly from) compartments 376. This arrangement provides a greater area of surface contact between hydrogel 358 and inner membrane layer 352 than if scaffold 360 intervened more between the hydrogel and the inner membrane layer. Such greater surface contact provides greater exchange of oxygen and other nutrients between the hydrogel and the membrane. This arrangement also helps scaffold 360 hold outer hydrogel layer 356 attached to inner membrane layer 352, because second portion 370 of hydrogel 358, which is integral with first portion 362 of hydrogel 358, is directly below (inwardly from) lateral walls 374 of scaffold 360 (described hereinbelow). In other words, hydrogel 358 is typically a single contiguous mass that is shaped so as to define first and second portions 362 and 370. Typically, an average distance between inner surface 366 of scaffold 360 and outer surface 364 of inner membrane layer 352 is at least 20 microns (e.g. at least 40 microns), no more than 300 microns (e.g., no more than 100 microns), and/or between 20 and 300 microns (e.g., between 40 and 100 microns).

For some applications, at least a portion 372 of inner surface 368 of scaffold 360 is disposed in direct contact with second portion 370 of hydrogel 358, and has a first surface area. Outer surface 364 of inner membrane layer 352 coated by outer hydrogel layer 356 has a second surface area. Typically, the first surface area equals at least 5%, no more than 30%, and/or between 5% and 30% of the second surface area.

For some applications, scaffold 360 is shaped so as to define a plurality of lateral walls 374, which, for some applications, are arranged as a network of rigid support bars, which may be arranged as a grid, as shown in FIGS. 11A-12D, or in another non-grid geometry. For some of these applications, lateral walls 374 define a plurality of compartments 376, which are open at outer and inner sides (i.e., at the top and bottom.). First portion 362 of hydrogel 358 is disposed in compartments 376. For example, lateral walls 374 may define at least 4 compartments 376, no more than 20 compartments 376, and/or between 4 and 20 compartments 376, such as at least 4 compartments 376, no more than 10 compartments 376, and/or between 4 and 10 compartments 376, e.g., 6 compartments 376, as shown in the figures. For some applications, each of compartments 376 has a surface area (i.e., a size of the top opening) of at least 0.25 mm2 (e.g., 0.5 mm×0.5 mm), no more than 4 mm2, and/or between 0.25 and 4 mm2. For example, in configurations in which compartments 376 are rectangular, each may have a length of between 1 and 3 mm, and/or a width of between 0.25 and 1.5 mm. Alternatively or additionally, for some applications, lateral walls 374 (and thus compartments 376) have an average height of at least 25 microns, no more than 300 microns, and/or between 25 and 300 microns, e.g., between 100 and 250 microns, e.g., 200 microns. For some applications, lateral walls 374 include peripheral lateral walls 374A and internal lateral walls 374B. For some applications, internal lateral walls 374B have an average height H (labeled in FIG. 12B) of at least 1 micron, no more than 300 microns, and/or between 1 and 300 microns, e.g., at least 20 microns, no more than 200 microns (e.g., no more than 100 microns), and/or between 20 and 200 microns (e.g., between 20 and 100 microns). Alternatively or additionally, for some applications, internal lateral walls 374B have an average thickness T (labeled in FIG. 12B) of at least 50 micron (e.g., at least 100 microns), no more than 300 microns (e.g., no more than 200 microns), and/or between 50 and 300 microns (e.g., between 100 and 200 microns).

For other applications, lateral walls 374 define a single compartment having relatively narrow portions, e.g., maze-shaped, serpentine, S-shaped, zigzag, etc. (configuration not shown).

For some applications, whether lateral walls 374 define a plurality of compartments 376 or a single compartment, a largest circular disc that can fit between lateral walls 374, while the circular disc is oriented parallel to inner membrane layer 352, has a diameter of at least 0.5 mm, no more than 3 mm, and/or between 0.5 and 3 mm. It is to be understood that the circular disc is not an element of the device, but rather an abstract shape used to describe a geometric property of the device.

For some applications, particularly in those application in which scaffold 360 is shaped so as to define lateral walls 374, an outer (upper) surface 380 of upper hydrogel layer 356 is disposed between 50 microns inwardly from (below) and 50 microns outwardly from (above) an outer (upper) surface 382 of scaffold 360, e.g., is disposed flush with outer surface 382 of scaffold 360 (labeled in FIG. 12C). This arrangement may further minimize the likelihood of outer hydrogel layer 356 peeling off of frame 100.

Typically, scaffold 360 is more rigid than hydrogel 358. For some applications, scaffold 360 comprises polysulfone, polyether sulfone, poly sulfone, PMMA, polypropylene, polyethylene (HDPE), PEI, PTFE, COC, and/or a combination of two or more of these materials. Typically, scaffold 360 does not comprise any gel. For some applications, scaffold 360 is manufactured by laser cutting.

Reference is made to FIGS. 11A and 12D. For some applications, as mentioned above with reference to FIGS. 1A-C and 2A-B, chamber 130 is shaped so as to define two or more sub-chambers. For example, chamber 130 may comprise:
a cell compartment 384, in which the cells are disposed, e.g., encapsulated, possibly attached to a solid scaffold material, e.g., comprising microcarrier beads, fibers, a rigid structure, a sponge structure, or another type of solid scaffold, or alternatively disposed on the walls of cell compartment 384; typically, injection opening 122, described hereinabove with reference to FIG. 5, opens into cell compartment 384, and the cells, possibly attached to the above-mentioned solid scaffold material, are injected through injection opening 122 into cell compartment 384; and
a biosensor compartment 386, which is typically free of cells and contains primarily secreted biosensor protein that diffuses from cell compartment 384; providing a separate biosensor compartment may allow a more accurate measurement, minimize blocking of light by the cells, and/or minimize exposure of the cells and biosensor protein still not secreted from the cells to excitation light.

For some applications, chamber 130 comprises a partial barrier 388 that partially separates cell compartment 384 from biosensor compartment 386, typically so as to inhibit passage of the cells from cell compartment 384 from biosensor compartment 386 (e.g., by preventing passage of the solid scaffold material, e.g., microcarrier beads, to which the cells are attached). On the other hand, partial barrier 388 allows passage of the secreted biosensor protein from cell compartment 384 from biosensor compartment 386. For example, partial barrier 388 may comprise a plurality of pillars (poles) 392, which are sized and shaped to prevent the passage of the solid scaffold material, e.g., microcarrier beads, to which the cells are attached. The shape of poles 392, distance between poles 392, and/or the vertical distance between poles 392 and inner membrane layer 352 are configured, for example, such that the microcarrier heads (which are typically larger than 100 microns and smaller than 300 microns, e.g., 200 microns), cannot pass through to biosensor compartment 386.

For some applications, frame 100 is shaped so as to define elevated areas 394 around the edges of scaffold 360. For some applications, elevated areas 394 are configured to be melted by a combination of heat and pressure in order to attach scaffold 360 to frame 100. Elevated areas 394 are typically between 50 and 150 microns higher than other parts of the top surface of frame 100.

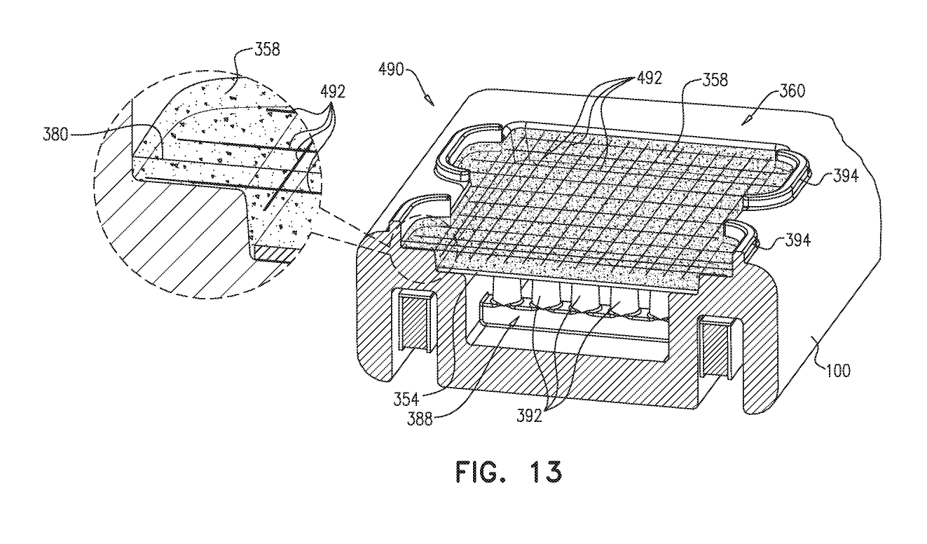

Reference is now made to FIG. 13, which is a schematic cross-sectional illustration of an implantable immunoisolation device 490 for encapsulation of live cells in a body of a subject, in accordance with an application of the present invention. Except as described hereinbelow, implantable immunoisolation device 490 is the same as implantable immunoisolation device 390, described hereinabove with reference to FIGS. 11A-12D, and may implement any of the features thereof, mutatis mutandis.

In this configuration, scaffold 360 comprises a porous structure 492, which may comprise, for example, a mesh, a net, or a fabric, similar in some respects to multi-layer immunoisolation system 500, described hereinabove with reference to FIG. 10. Typically, in this configuration, scaffold 360 is disposed inwardly from (below) outer surface 380 of outer hydrogel layer 356, for example between 50 microns inwardly from (below) outer surface 380 and 50 microns outwardly from (above) membrane 354, and/or between a distance inwardly from (below) outer surface 380 and a distance outwardly from (above) membrane 354, the distance equal to 10% of an average height of outer hydrogel layer 356. Alternatively, scaffold 360 is flush with outer surface 380 of outer hydrogel layer 356.

As used in the present application, including in the claims, “outer” means at or toward an external surface of the immunoisolation device, and “inner” means toward a center of the immunoisolation device. The words “upper” and “lower” have also been used to provide relative directions based on the orientation of the immunoisolation devices in the figures; “upper” and “lower” correspond with “outer” and “inner,” respectively, given the orientation of the immunoisolation devices in the figures.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
U.S. Pat. No. 7,951,357 to Gross et. al.;
U.S. Pat. No. 9,037,205 to Gil et. al.;
US Patent Application Publication 2010/0160749 to Gross et al.;
US Patent Application Publication 2010/0202966 to Gross et al.;
US Patent Application Publication 2011/0251471 to Gross et al.;
US Patent Application Publication 2012/0059232 to Gross et al.;
US Patent Application Publication 2015/0343093 to Hyman et al.;
US Patent Application Publication 2015/0352229 to Brill et al.;
US Patent Application Publication 2016/0324449 to Gross et al.;
PCT Publication WO 2006/006166 to Gross et al.;
PCT Publication WO 2007/110867 to Gross et al.;
PCT Publication WO 2010/073249 to Gross et al.;
PCT Publication WO 2013/001532 to Gil et al.;
PCT Publication WO 2014/102743 to Brill et al.;
PCT Publication WO 2015/128826 to Barkai et al.;
PCT Publication WO 2016/059635 to Brill;
U.S. Provisional Patent Application 60/588,211, filed Jul. 14, 2004;
U.S. Provisional Patent Application 60/658,716, filed Mar. 3, 2005;
U.S. Provisional Patent Application 60/786,532, filed Mar. 27, 2006;
U.S. Provisional Patent Application 61/149,110, filed Feb. 2, 2009;
U.S. Provisional Patent Application 61/746,691, filed Dec. 28, 2012;
U.S. Provisional Patent Application 61/944,936, filed Feb. 26, 2014; and
U.S. Provisional Patent Application 62/063,211, filed Oct. 13, 2014.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for implantation of live cells in a subject, the apparatus comprising an implantable immunoisolation device, which comprises:
a chamber, which contains the live cells;
an inner membrane layer, which is disposed at an external surface of the chamber, and which comprises a selective membrane that is permeable to nutrients; and
an outer hydrogel layer, which comprises a hydrogel, and which is attached to and coats an outer surface of the inner membrane layer.

2. The apparatus according to claim 1, wherein the implantable immunoisolation device further comprises a non-biodegradable scaffold, and wherein a portion of the hydrogel is disposed in the scaffold, such that the scaffold helps hold the outer hydrogel layer attached to the outer surface of the inner membrane layer.

3. The apparatus according to claim 2,
wherein at least a portion of an inner surface of the scaffold is disposed over the inner membrane layer,
wherein at least 75% of the at least a portion of the inner surface of the scaffold is a non-contacting inner surface that does not directly contact the outer surface of the inner membrane layer,
wherein the portion of the hydrogel disposed in the scaffold is a first portion of the hydrogel, and
wherein a second portion of the hydrogel is disposed between a height of the non-contacting inner surface and the outer surface of the inner membrane layer.

4. The apparatus according to claim 3, wherein 100% of the inner surface of the scaffold does not directly contact the outer surface of the inner membrane layer.

5. The apparatus according to claim 3, wherein an average distance between the inner surface of the scaffold and the outer surface of the inner membrane layer is between 20 and 300 microns.

6. The apparatus according to claim 3,
wherein at least a portion of an inner surface of the scaffold is disposed in direct contact with the second portion of the hydrogel, and has a first surface area,
wherein the outer surface of the inner membrane layer coated by the outer hydrogel layer has a second surface area, and
wherein the first surface area equals between 5% and 30% of the second surface area.

7. The apparatus according to claim 2,
wherein the implantable immunoisolation device comprises a frame, which is shaped so as to define the chamber, and
wherein the scaffold is attached to the frame.

8. The apparatus according to claim 2, wherein the scaffold is shaped so as to define a plurality of lateral walls.

9. The apparatus according to claim 8, wherein the lateral walls define a plurality of compartments, which are open at outer and inner sides, and wherein the portion of the hydrogel is disposed in the compartments.

10. The apparatus according to claim 9, wherein the lateral walls define between 4 and 20 compartments.

11. The apparatus according to claim 9, wherein each of the compartments has a surface area of between 0.25 mm2 and 4 mm2.

12. The apparatus according to claim 8, wherein the lateral walls have an average height of between 25 and 300 microns.

13. The apparatus according to claim 8, wherein a largest circular disc that can fit between the lateral walls, while the circular disc is oriented parallel to the inner membrane layer, has a diameter of between 0.5 and 3 mm.

14. The apparatus according to claim 2, wherein an outer surface of the outer hydrogel layer is disposed between 50 microns inwardly from and 50 microns outwardly from an outer surface of the scaffold.

15. The apparatus according to claim 14, wherein the outer surface of the outer hydrogel layer is disposed flush with the outer surface of the scaffold.

16. The apparatus according to claim 2, wherein the scaffold is more rigid than the hydrogel.

17. The apparatus according to claim 2, wherein the scaffold comprises a porous structure.

18. The apparatus according to claim 17, wherein the porous structure comprises an element selected from the group consisting of: a mesh, a net, and a fabric.

19. The apparatus according to claim 2, wherein the scaffold is fixed to the inner membrane layer.

20. The apparatus according to claim 1,
wherein the implantable immunoisolation device comprises a frame, which is shaped so as to define the chamber, and
wherein the outer hydrogel layer is flush with an outer surface of the frame at least partially along an interface between the outer hydrogel layer and the frame.

21. The apparatus according to claim 20, wherein the outer hydrogel layer is flush with the outer surface of the frame along at least 10% of a length of the interface between the outer hydrogel layer and the frame.

22-24. (canceled)

25. The apparatus according to claim 1, wherein the implantable immunoisolation device comprises a frame, which is shaped so as to define the chamber, and wherein the inner membrane layer is directly connected to the frame with no intermediate material.

26. The apparatus according to claim 1, wherein the apparatus comprises a frame, which is shaped so as to define the chamber, and wherein the inner membrane layer is directly connected to the frame without being glued to the frame.

27-28. (canceled)

29. The apparatus according to claim 1, wherein the hydrogel is non-biodegradable.

30. The apparatus according to claim 1, wherein the outer hydrogel layer covers at least 50% of the external surface of the inner membrane layer.

31-39. (canceled)

40. The apparatus according to claim 1,
wherein the implantable immunoisolation device comprises a frame, which is shaped so as to define the chamber, and
wherein side walls of the frame that extend along a longest dimension of the frame are inclined.

41-58. (canceled)
